(19) United States
(12) Patent Application Publication
(10) Pub. No.: US 2009/0181375 A1
(43) Pub. Date: Jul. 16, 2009

(54) METHOD FOR DETECTION OF NUCLEIC ACID BARCODES

(76) Inventors: Brian J. Peter, Los Altos, CA (US); Joel Myerson, Berkeley, CA (US); Robert A. Ach, San Francisco, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/013,378

(22) Filed: Jan. 11, 2008

(51) Int. Cl. C12Q 1/68; C40B 40/06

(52) U.S. Cl. 435/6; 506/16

(57) ABSTRACT

A method of sample analysis is provided. In certain embodiments, the method may comprise: a) contacting a surface-tethered oligonucleotide with a sample comprising a barcode oligonucleotide to produce an oligonucleotide duplex comprising a double-stranded surface-proximal region and a single-stranded surface-distal overhang; b) extending the barcode oligonucleotide using the overhang as a template to produce an extended duplex; c) subjecting the extended duplex to a wash that separates the oligonucleotide duplex but does not separate said extended duplex; and d) detecting the extended duplex.

### BACKGROUND

In the face of remarkable developments in the fields of molecular biology and genetics in the past few decades, it has become increasingly important to develop tools to use in a variety of research, medical, and industrial applications, such as identifying disease-related polynucleotides, screening for novel targets, DNA sequencing, amplification of target polynucleotides, etc. Detection of specific hybridization of oligonucleotides to their complements has been employed as such tools in many fields. The oligonucleotides serve as barcode oligonucleotides to track, identify, and retrieve biomolecules of interest.

This disclosure relates to a method in detecting barcode oligonucleotides.

### SUMMARY

A method of sample analysis is provided. In certain embodiments, the method may comprise: a) contacting a surface-tethered oligonucleotide with a sample comprising a barcode oligonucleotide to produce an oligonucleotide duplex comprising a double-stranded surface-proximal region and a single-stranded surface-distal overhang; b) extending the barcode oligonucleotide using the overhang as a template to produce an extended duplex; c) subjecting the extended duplex to a wash that separates the oligonucleotide duplex but does not separate the extended duplex; and d) detecting the extended duplex.

### BRIEF DESCRIPTION OF THE FIGURES

FIG. 1 schematically illustrates a surface-tethered oligonucleotide and an overhang duplex.

FIG. 2 schematically illustrates certain features of some embodiments of a method described herein.

### DEFINITIONS

The term “sample” as used herein relates to a material or mixture of materials, typically, although not necessarily, in liquid form, containing one or more analytes of interest.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide” as used herein denotes a single stranded multimer of nucleotide of from about 2 to 200 nucleotides. Oligonucleotides may be synthetic or may be made enzymatically, and, in some embodiments, are under 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200 nucleotides in length, for example.

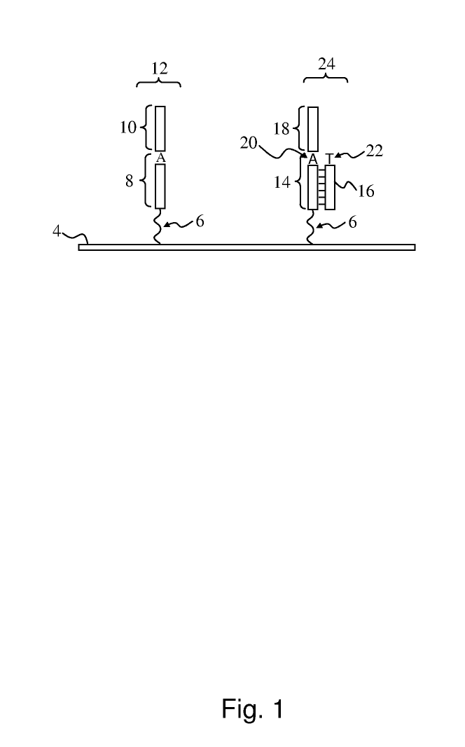

The term “barcode oligonucleotide” as used herein refers to an oligonucleotide that has a nucleotide sequence that uniquely identifies a target analyte nucleic acid in a sample. In certain cases, a barcode oligonucleotide may hybridize to a target nucleic acid in a sample. In other embodiments, a barcode oligonucleotide may be a cleavage product of a longer polynucleotide. A barcode oligonucleotide is indicated as element 16 in the schematic illustrations of FIG. 1.

The term “a surface-tethered oligonucleotide” as used herein refers to a nucleic acid that is immobilized on a surface of a substrate, where the substrate can have a variety of configurations, e.g., a sheet, bead, or other structure. In certain embodiments, a surface-tethered oligonucleotides may be present on a surface of a planar support, e.g., in the form of an array. A surface-tethered oligonucleotide is indicated as element 12 in the schematic illustration of FIG. 1.

The term “oligonucleotide duplex” as used herein refers to a duplex formed by hybridization of two oligonucleotides containing complementary sequences, e.g. a barcode oligonucleotide and a surface-tethered oligonucleotide. An oligonucleotide duplex is indicated as element 24 in the schematic illustration of FIG. 1.

The terms “surface-proximal region” and “surface-distal region” are relative terms that refer to portions of a surface-tethered oligonucleotide that are proximal or distal to the surface to which the oligonucleotide is tethered. The surface-proximal and surface distal regions of an oligonucleotide are indicated in FIG. 1 as element 8 and 10, respectively.

The term “extending” as used herein refers to any addition of one or more nucleotides to the end of a nucleic acid, e.g. by ligation of an oligonucleotide or by using a polymerase.

As used herein, the term “overhang” refers to a single-stranded region of a duplex containing two oligonucleotides, where one of the oligonucleotides comprises additional nucleotides in addition to the complementary region. An overhang may be surface-distal or surface-proximal. An overhang of a duplex is indicated in FIG. 1 as element 18.

As used herein, the term “overhang duplex” refers to a duplex, e.g., an oligonucleotide duplex that contains an overhang. An overhang duplex is indicated in FIG. 1 as element 24.

As used herein, in the context of overhang duplex, the term “overhang-adjacent nucleotide”, refers to a terminal nucleotide of an oligonucleotide that lies immediately adjacent to the overhang of an overhang duplex. The overhang-adjacent nucleotide of a duplex is indicated in FIG. 1 as element 22.

The term “corresponding nucleotides” as used herein refers to nucleotides in a nucleic acid duplex that are positioned directly across from each other. Corresponding nucleotides may be base paired or not base-paired with each other. In the context an overhang duplex, the nucleotide that corresponds to an overhang-adjacent nucleotide is the nucleotide positioned directly across from overhang-adjacent nucleotide 16. Such a nucleotide may be base-paired with or not base paired with the overhang-adjacent nucleotide. A nucleotide that corresponds to the overhang-adjacent nucleotide of an overhang duplex is indicated as element 20 of FIG. 1.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

As used herein, the term “Tₘ” refers to the melting temperature an oligonucleotide duplex at which half of the duplexes remain hybridized and half of the duplexes dissociate into single strands. The Tₘ of an oligonucleotide duplex may be experimentally determined or calculated using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10).

As used herein, the term “Tₘ-matched” refers to a plurality of nucleic acid duplexes having Tₘs that are within a defined range.

The term “low-stringency hybridization conditions” as used herein refers to hybridization conditions that are suitable for hybridization of a barcode oligonucleotide and a surface-tethered oligonucleotide that has a region that is complementary to the barcode oligonucleotide. Such conditions may differ from one experiment to the next depending on the length and the nucleotide content of the complementary region. In certain cases, the temperature for low-stringency hybridization is 5°-10° C. lower than the calculated Tₘ of the resulting duplex under the conditions used.

As used herein “high-stringency wash conditions” refers to wash conditions that provide for disassociation of non-extended duplexes that contain non-extended barcode oligonucleotides, but not disassociation of extended duplexes with extended barcode oligonucleotides. Such conditions release barcode oligonucleotides that are not extended from the surface-tethered oligonucleotide but do not release extended barcode oligonucleotides from the surface-tethered oligonucleotide. Again, such conditions may differ from one experiment to the next depending on the length and the nucleotide content of the complementary region. In certain cases, the temperature for a high stringency wash may be 5°-10° C. lower than the calculated Tₘ of an extended duplex, and 5°-10° C. higher than the calculated Tₘ of a non-extended duplex, under the conditions used.

As used herein, the term “single nucleotide polymorphism”, or “SNP” for short, refers to single nucleotide position in a genomic sequence for which two or more alternative alleles are present at appreciable frequency (e.g., at least 1%) in a population.

As used herein, the term “SNP nucleotide” refers to a nucleotide that is the same as or complementary to a SNP. In certain embodiments, a SNP nucleotide is the terminal nucleotide in a barcode oligonucleotide, and is used to identify the SNP in a target.

As used herein, the term “variable region” in the context of an array of oligonucleotides refers to a region of the oligonucleotides where the nucleotide sequence varies from oligonucleotide to oligonucleotide.

As use herein, the term “constant region” in the context of an array of oligonucleotides refers to a region of the oligonucleotides that has a nucleotide sequence that does not vary from oligonucleotide to oligonucleotide. Constant regions of a plurality of oligonucleotides contain the same nucleotide sequence.

As used herein, the term “overlap-dependent cleavage assay” refers to an assay in which a subject polynucleotide is cleaved to release a barcode oligonucleotide where cleavage only occurs when there are overlapping oligonucleotides in a complementary region.

### DESCRIPTION OF EXEMPLARY EMBODIMENTS

A method of sample analysis is provided. In certain embodiments, the method may comprise: a) contacting a surface-tethered oligonucleotide with a sample comprising a barcode oligonucleotide to produce an oligonucleotide duplex comprising a double-stranded surface-proximal region and a single-stranded surface-distal overhang; b) extending the barcode oligonucleotide using the overhang as a template to produce an extended duplex; c) subjecting the extended duplex to a wash that separates the oligonucleotide duplex but does not separate the extended duplex; and d) detecting the extended duplex.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method for Detecting Barcode Oligonucleotides

In general terms, the subject method includes contacting a surface-tethered oligonucleotide with a sample comprising a barcode oligonucleotide under hybridization conditions to provide for the hybridization of the barcode oligonucleotide and the surface-tethered oligonucleotide. The method further includes extending the barcode oligonucleotide to produce an extended duplex, subjecting the extended duplex to conditions that provide for its separation from the non-extended duplex (e.g. washing), and detecting the extended duplex.

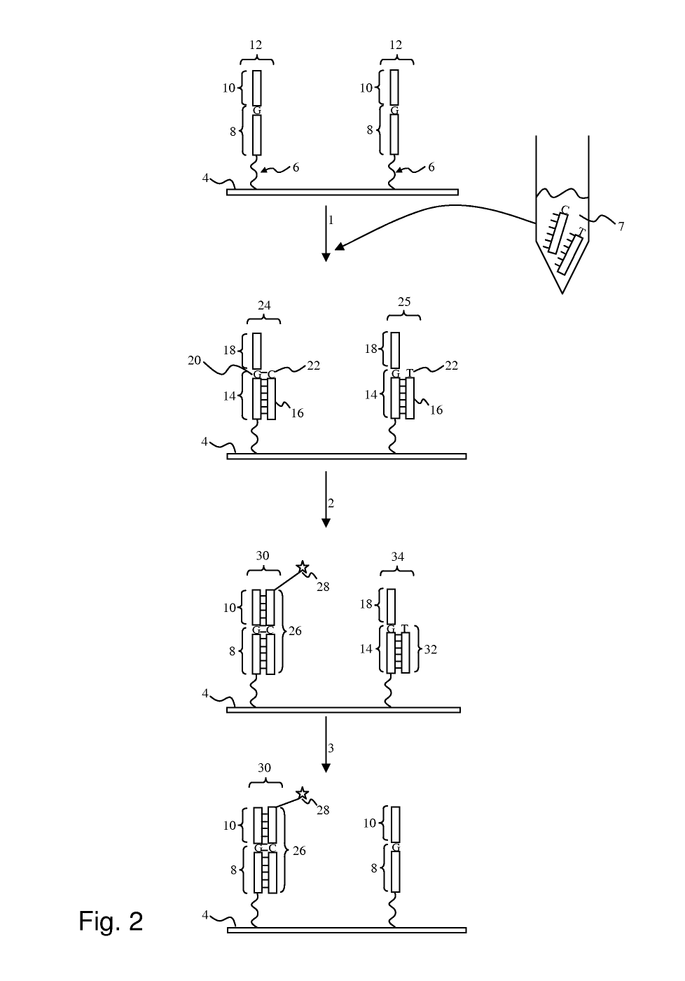

Certain features of the subject method are illustrated in FIG. 2 and are described in greater detail below. With reference to FIGS. 1 and 2, the method generally includes contacting 1 surface-tethered oligonucleotide 12 with sample 7 containing barcode oligonucleotides 16 under hybridization conditions to provide overhang duplexes 24 and 25. The duplexes are then extended 2 using overhang 18 of the surface-tethered oligonucleotide as the template. If there is sufficient complementary between the surface-tethered oligonucleotide and the barcode oligonucleotide, as in duplex 24, the barcode oligonucleotide is be extended to increase stability and Tₘ of a duplex. If there is insufficient complementary between the surface-tethered oligonucleotide and the barcode oligonucleotide, as in duplex 25, the barcode oligonucleotide is not extended and there is no change to the Tₘ of a duplex. The duplexes are then subjected to wash conditions 3 that provide for disassociation of the non-extended duplexes 34 but not the extended duplexes 30. Barcode oligonucleotides that are extended can then be detected.

In certain embodiments, with reference to FIG. 2, contacting 1 may produce oligonucleotide duplex 24 comprising double-stranded surface-proximal region 14 and single-stranded surface-distal overhang 18. A single-stranded overhang is made up of additional nucleotides on the surface-tethered oligonucleotide beyond the region that is complementary to the barcode oligonucleotide.

The contacting step of the method is generally performed under conditions suitable for annealing of a barcode oligonucleotide to a surface-tethered oligonucleotide to produce an oligonucleotide duplex. As noted above, while such hybridization conditions may vary depending on the length and composition of the region of complementarity between the two oligonucleotides, suitable conditions are nevertheless known and described in, e.g., Sambrook et al, supra. In certain cases, conditions suitable for successful hybridization of a barcode oligonucleotide and a surface-tethered oligonucleotide may be determined by calculating the Tₘ of the expected oligonucleotide duplex in a particular hybridization buffer using the formula Tₘ=81.5+16.6(log₁₀[Na⁺])+0.41 (fraction G+C)−(60/N), where N is the chain length and [Na⁺] is less than 1 M. In these cases, the hybridization temperature may be 2°-10° C., e.g., 5°-10° C., lower than the calculated Tₘ of the expected oligonucleotide duplex. Suitable hybridization conditions may also be determined experimentally.

After oligonucleotide duplex 24 is formed between surface-tethered oligonucleotide 12 and barcode oligonucleotide 16, the duplex is subjected to template-dependent extension 2 using overhang 18 as the template, as illustrated in FIG. 2. In certain embodiments, a polymerase may be employed to add nucleotides, e.g., labeled nucleotides to the 3′ end of the barcode oligonucleotide. In other cases, a ligase may be used to ligate an oligonucleotide, e.g. labeled oligonucleotides to an end of the barcode oligonucleotide. By extending the oligonucleotide duplex, the length of double-stranded region 14 is increased. Consequently, the Tₘ of extended duplex 30 is higher than the Tₘ of the duplex before extension or non-extended duplex 34. Further, extension 2 may also incorporate a label 28 into the oligonucleotide duplex for subsequent detection.

In certain cases, the oligonucleotide duplex may contain regions that are not complementary, and, as such, may not be extended despite being subjected to extension conditions. The non-extended duplex may be, for example, a duplex comprising a surface-tethered oligonucleotide and a barcode oligonucleotide that are not fully complementary, or a duplex in which the overhang-adjacent nucleotide is not complementary to the corresponding nucleotide in the surface-tethered nucleotide. For example, as illustrated in FIG. 2, if a sample contains barcode oligonucleotide 16 that is not perfectly matched to surface-proximal region 8 of the surface-tethered oligonucleotide, such an oligonucleotide duplex formed by imperfectly matched oligonucleotides may not be extended. In another example, some barcode oligonucleotides 16 may include nucleotides beyond overhang-adjacent nucleotide 22 which, if they are not complementary to overhang 18, may not be extended.

After extension 2, the duplex is subjected to wash conditions 3 that separate non-extended barcode oligonucleotides, but not extended barcode nucleotides, from the surface-tethered oligonucleotide. In certain cases, the wash comprises conditions that preferentially allow separation of the oligonucleotide duplex as compared to the extended duplex. Since extension of the barcode oligonucleotide exclusively increases the Tₘ of duplexes in which the barcode oligonucleotides are extended, extended barcode oligonucleotides 26 and non-extended barcode oligonucleotides 32 can be discriminated. Only duplexes that have extended barcode oligonucleotides 26 will remain intact after washing and are detected by detecting the incorporated label 28. Since the Tₘ is increased for extended duplex 30 compared to the Tₘ of non-extended duplex 34, the wash conditions are at a stringency that is higher than the hybridization conditions used. In certain embodiments, the temperature of the wash may be chosen so that it is 2′-10° C., e.g., 5′-10° C. lower than the Tₘ of an extended duplex but 2′-10° C., e.g., 5′-10° C. higher than the Tₘ of the non-extended duplex, under the conditions used. As would be recognized by one of skill in the art, in certain cases the wash temperature may be higher than the hybridization temperature, e.g., by at least 5° C., at least 10° C. or at least 20° C., up to about 30° C. In other cases, the concentration of ions, e.g., Na⁺ in the wash buffer may be less than the concentration of ions in the hybridization buffer, e.g., by at least 50%, at least 80%, at least 90% or up to about 95%. In other embodiments, the wash may be done in a buffer containing less ions and at a lower temperature than the hybridization. Such conditions are readily calculable using the following formula: Tₘ=81.5+16.6(log₁₀[Na⁺])+0.41 (fraction G+C)−(60/N), where N is the chain length and [Na⁺] is less than 1 M, where the wash and hybridization temperatures are 2°-10° C. lower than the calculated Tₘ for an extended duplex and non-extended duplex, respectively. In other embodiments, the stringency of the wash buffer may be altered by changing the concentration of a denaturant such as formamide. Such hybridization and wash conditions and reagents, e.g., SSC, SSPE, etc., for making the same are described in great detail in Sambrook, supra.

In one exemplary embodiment, the addition of 4 guanines raises the Tₘ of a 20-mer duplex by approximately 10° C. As such, the wash may be done at a temperature that is 4-6° C. less than the Tₘ of the extended duplex (which will be 4-6° C. higher than the Tₘ of the non-extended duplex).

The higher stringency of the wash conditions effectively separates non-extended duplexes from the barcode oligonucleotide in the non-extended duplexes. The extended duplexes do not disassociate. The selective disassociation of non-extended duplexes allows for detection of extended barcode oligonucleotides that are annealed to the surface-tethered oligonucleotides.

After subjecting the extended duplex to high-stringency wash conditions, the retained extended duplex may be detected by detecting a label, e.g. a fluorescent or a hapten label in the extended barcode oligonucleotide. In certain embodiments, the label may already be present in a pre-labeled barcode oligonucleotide or be incorporated during extension. For example, contacting an oligonucleotide duplex with a reagent mix containing polymerase and labeled nucleotides produces extended duplex 30 that is labeled. In certain embodiments, reagent mix comprises nucleotides of more than one type, in which one of the types of the nucleotides may be labeled and the other types are unlabeled. In these embodiments, the types of labeled and unlabeled nucleotides in the reagent mix could be chosen to control the number or type of labels added. In an embodiment, unlabeled nucleotides may extend the barcode oligonucleotide and the labeled nucleotide is added as the terminal nucleotide. For example, an overhang of an oligonucleotide duplex may comprise of a stretch of cytosines followed by a thymine as the terminal nucleotide. In this example, an extension reaction would comprise of unlabeled guanines to complement the stretch of cytosines and labeled adenosines to complement the terminal nucleotide. In another example, modified labeled nucleotides such as dideoxynucleotides could be used to ensure the addition of a single label per oligonucleotide duplex.

In another embodiment, a reagent mix comprises a labeled oligonucleotide to be ligated produces a labeled extended duplex. In this embodiment, the oligonucleotide to be ligated onto the ends of the barcode oligonucleotides may contain one or more labels. In a variation of this embodiment, the oligonucleotide may be of a branched structure so there are many placements for multiple labels.

In an alternative embodiment, the subject method may comprise extending a barcode oligonucleotide, contacting a surface-tethered oligonucleotide with the extended barcode oligonucleotide to produce an extended duplex, and detecting the extended duplex. In this embodiment, the barcode oligonucleotide is extended before being contacted with the surface-tethered oligonucleotide. In these embodiments, the extension may be catalyzed by a terminal transferase or T4 RNA ligase in a template-independent fashion. In these embodiments, the barcode oligonucleotide may be extended using a single type of nucleotide to create a homopolymeric tract. For example, either adenine or thymine could be used. If cytosine is chosen to be the nucleotides to be used in the extension reaction in this embodiment, there may be no more than 4 guanines on the surface-tethered oligonucleotide beyond the region complementary to the barcode oligonucleotide to avoid secondary structures caused by poly-guanine tracts. In an embodiment, chain-terminating nucleotides such as dideoxynucleotides could be included in the extension reaction. Template-independent extension of barcode oligonucleotide may also effectively increase the Tₘ of the resulted duplex after contacting the extended barcode oligonucleotide with the surface-tethered oligonucleotide.

The polymerase used in the extension reaction may include but not limited to DNA polymerase, such as a template-dependent polymerase (e.g., T4 DNA polymerase, Taq polymerase, reverse transcriptase (MMLV RT), the Klenow fragment of DNA polymerase I and the like), Pfu, or a template-independent DNA polymerase (such as terminal transferase), a polynucleotide ligase (such as T4 DNA ligase, etc.), or any combination thereof. In certain cases it may be advantageous to employ a polymerase without a 5′ to 3′ exonuclease (“proofreading”) activity. As would be recognized by one of skill in the art, a wide variety of DNA polymerases and ligases employable in the subject methods are available.

In certain embodiments, the barcode oligonucleotides may comprise modifications such as phosphorothioate linkages, isotopic or fluorescent 3′ residues, 3′ phosphorylation, 3′ dehydroxylation, etc., to render the oligonucleotides resistant to exonuclease digestion or polymerase extension. In embodiments, one subset of the barcode oligonucleotides may comprise certain modifications while another subset does not, allowing discrimination of the two subsets based on their resistance to extension or degradation.

In certain embodiments, with reference to FIGS. 1 and 2, the surface-tethered oligonucleotide may be linked to planar surface 4 of a substrate such as a region of an array substrate, or to a bead, which may provide for the isolation of the surface-tethered oligonucleotides and their binding partners. In certain embodiments, surface-tethered oligonucleotide 12 has surface-proximal region 8 complementary to a barcode oligonucleotide, where the surface proximal region is of a length in a range of 10-30 nucleotides, e.g., 10-20, 15-30, or more nucleotides. In certain cases, the surface-tethered oligonucleotide has at least 4, e.g., 4-10, up to 20 or more additional nucleotides beyond the barcode oligonucleotide complementary region as surface-distal region 10. In certain embodiments and as will be described in greater detail below, the terminal nucleotide of a barcode oligonucleotide may be a SNP nucleotide. In these embodiments, the surface-tethered oligonucleotide may comprise corresponding nucleotide 20 that is complementary to the SNP-nucleotide as the terminal nucleotide of the complementary region 8.

In an array comprising a plurality of surface-tethered oligonucleotides, surface-proximal region 8 may be a variable region that is complementary to barcode oligonucleotides of different nucleotide sequences. The surface-distal region 10 of a surface-tethered oligonucleotide may be a constant region. In an example with reference to FIG. 2, surface-proximal regions 8 of the two surface-tethered oligonucleotides may be of different sequences while their surface-distal regions 10 may be of the same nucleotide sequence.

In additional embodiments, the nucleotides in surface-distal region may be rich in guanine and cytosine to maximize stability and Tₘ of the duplex after extension. In certain embodiments, these nucleotides may be of a single type comprising a homopolymeric tract in the surface-tethered oligonucleotides. For example, a surface-tethered oligonucleotide may comprise 2,6-aminopurines in the surface-distal region to avoid secondary structures formed in the surface distal region. The base-pair containing 2,6-aminopurines may also have increased stability and Tₘ compare to the stability and the Tₘ of traditional A::T and G:::C pairings. In this example, extension of the barcode oligonucleotide may require only thymines as the nucleotides.

In certain embodiments, a barcode oligonucleotide may be of a length of at least 5-50 nucleotides, e.g., 5-20, 5-10, 11-20, 21-50 or more nucleotides. A barcode nucleotide may be labeled or unlabeled. Because the instant method comprises of an extension step where labels may be incorporated, the choice of barcode oligonucleotides may be flexible due to the fact that they need not be labeled.

In certain cases, the sample may contain as few as one barcode oligonucleotide. However, in other embodiments, the sample may contain at least 2, at least 4, at least 10, at least 100 or at least 1,000, up to at least 10,000 barcode nucleotides. In certain embodiments, a sample may contain a plurality of barcode oligonucleotides of the same nucleotide sequence or of different nucleotide sequences. Further, the barcodes oligonucleotides of a sample may be for detecting single-nucleotide polymorphisms (SNPs), in which case the sample may contain a plurality of barcode oligonucleotides, where each barcode oligonucleotide identifies a different SNP.

In certain embodiments, the barcode oligonucleotide is generated from a cleavage reaction from a larger polynucleotide, where the cleavage is specific to the presence of a target analyte. In this embodiment, detection of the barcode may indicate the presence of the target analyte.

For example, in certain cases, the barcode is a product of an overlap-dependent cleavage reaction, where the presence of a barcode oligonucleotide may indicate the presence of a specific SNP. In such a cleavage reaction, a flap endonuclease activity (provided by FEN1 or other suitable enzymes) cleaves to produce a barcode oligonucleotide from a larger polynucleotide only when a complex is formed with specific complementary regions between nucleic acids, in which these complementary regions comprises the SNP. If a particular SNP is absent, no complementary regions would be present in the complex and no barcode oligonucleotide would be produced. Such assays, which may be also known as INVADERS assays, are generally known in the art and are described in detail in Mast et al. (Mast et al. “INVADER® Assay for Single-Nucleotide Polymorphism Genotyping and Gene Copy Number Evaluation.” Methods in Mol. Biol. (2006) 335:173-186), and Stevens et al. (Stevens et al. “Analysis of single nucleotide polymorphisms with solid phase invasive cleavage reactions.” Nucleic Acids Res. (2001) 29:e77).

In certain embodiments, this overlap-dependent cleavage produces a barcode oligonucleotide that contains an SNP nucleotide as its terminal nucleotide, which, with reference to FIGS. 1 and 2, may be overhang-adjacent nucleotide 22 when barcode oligonucleotide 16 is hybridized to surface-tethered oligonucleotide 12. With reference to the SNP nucleotide, the nucleotide on the surface-tethered oligonucleotide that lies directly across from overhang-adjacent nucleotide 22 is corresponding nucleotide 20.

### Array-Based Detection of Barcode Oligonucleotides

Certain embodiments of the subject method described herein provide an array comprising a set of surface-tethered oligonucleotides for detecting a plurality of barcode oligonucleotides. In general terms, the array-based detection method includes contacting 1 an array of surface-tethered oligonucleotides with a sample comprising barcode oligonucleotides to produce a template array comprising overhang duplexes, extending 2 the barcode oligonucleotides annealed to the surface-tethered oligonucleotides, subjecting the array to a wash 3 to disassociate the non-extended barcode nucleotides but not the extended barcode nucleotides, and reading the array to detect extended duplexes.

Each of the surface-tethered oligonucleotides on an array may comprise a surface-proximal region that has a nucleotide sequence that varies from oligonucleotide to oligonucleotide and that is complementary to a different barcode oligonucleotide. In addition, each of the surface-tethered oligonucleotides may comprise a surface-distal region comprising additional nucleotides beyond the region complementary to a barcode oligonucleotide. The nucleotide sequence of this region may be constant.

In certain embodiments, with reference to FIG. 2, contacting an array to a sample comprising barcode oligonucleotides produces a template array of overhang duplexes 24, each of which comprises double-stranded variable region 14 and single-stranded overhang 18 comprising additional nucleotides on the surface-tethered oligonucleotide beyond the region complementary to the barcode oligonucleotide.

In certain cases, surface-distal overhangs are constant from oligonucleotide to oligonucleotide, such that they may comprise the same nucleotides. In these embodiments, extension of the barcode oligonucleotides of the overhang duplexes on a template array may use the same nucleotides. In certain embodiments, barcode oligonucleotides to be detected in a sample are not of equal lengths. In such cases, an array of surface-tethered oligonucleotides may be designed to be Tₘ-matched, in that the extension of overhang duplexes would produce extended duplexes of similar melting temperature (e.g., within 1° or 2° C. of a chosen Tₘ) under the hybridization or washing conditions used. The Tₘ of a duplex may be calculated using conventional methods, e.g., in silico or experimentally. In this embodiment, the array may be subjected to one hybridization or washing condition that separates non-extended barcode oligonucleotides from extended barcode oligonucleotides. This embodiment may also allow the detection of barcode oligonucleotides of different lengths using one array.

In certain embodiments, the barcode oligonucleotides are products of a cleavage reaction specific to detect a certain SNP. In certain cases, each of the barcode oligonucleotides may comprise a terminal nucleotide as the SNP nucleotide. After contacting an array of surface-tethered oligonucleotides to produce a template array of overhang duplexes, the SNP nucleotides are overhang-adjacent nucleotides 22 of the overhang duplexes, with reference to FIG. 1. In certain embodiments, each of surface-tethered oligonucleotides on an array comprises of corresponding nucleotide 20 in a position that lie directly across from overhang-adjacent nucleotide 22 to complement a specific SNP nucleotide. In certain embodiments, complementarity between the corresponding nucleotides of the surface-tethered oligonucleotides and the overhang-adjacent nucleotides determines whether the barcode oligonucleotides can be extended. In certain cases, extension may indicate the presence of a SNP.

Since the nucleotide sequences of hundreds of thousand of SNPs from humans, other mammals (e.g., mice), and a variety of different plants (e.g., corn, rice and soybean), are known (see, e.g., Riva et al 2004, A SNP-centric database for the investigation of the human genome BMC Bioinformatics 5:33; McCarthy et al 2000 The use of single-nucleotide polymorphism maps in pharmacogenomics Nat Biotechnology 18:505-8) and are available in public databases (e.g., NCBI's online dbSNP database, and the online database of the International HapMap Project; see also Teufel et al 2006 Current bioinformatics tools in genomic biomedical research Int. J. Mol. Med. 17:967-73) the design of barcode oligonucleotides to identify SNP is well within the skill of one of skill in the art. The SNP may be known prior to design of a set of barcode oligonucleotides. The SNP may be linked to a phenotype (e.g., a disease) or may be unlinked to a phenotype (e.g., may be an “anonymous” SNP.

The subject arrays may contain a single set of features, e.g., a pair of features, one for each of a pair of surface-tethered oligonucleotides, for detecting a single SNP. However, in certain embodiments, the subject arrays may contain more than one such feature, and those features may correspond to (i.e., may be used to detect) a plurality of SNPs of a genome. Accordingly, the subject arrays may contain a plurality of features (i.e., 2 or more, about 5 or more, about 10 or more, about 15 or more, about 20 or more, about 30 or more, about 50 or more, about 100 or more, about 200 or more, about 500 or more, about 1000 or more, usually up to about 10,000 or about 20,000 or more features, etc.), each containing a different corresponding nucleotide to detect different SNPs. In certain embodiments, therefore, the subject arrays contain a plurality of oligonucleotide features that correspond to a plurality of SNPs of a genome. In particular embodiments, therefore, the subject arrays may contain features to detect, i.e., corresponding to, all of the predicted SNPs of a particular genome. The subject arrays may contain at least up to at least 45,000 different features to detect SNPs.

In general, arrays suitable for use in performing the subject method contain a plurality (i.e., at least about 100, at least about 500, at least about 1000, at least about 2000, at least about 5000, at least about 10,000, at least about 20,000, usually up to about 100,000 or more) of addressable features containing oligonucleotides that are linked to a usually planar solid support. In particular embodiments, SNPs of interest are represented by at least 2, about 5, or about 10 or more, e.g., up to about 20 sets of surface-tethered oligonucleotide features. Such an array may contain duplicate oligonucleotides or different surface-tethered oligonucleotides for the same SNP.

In a particular embodiment, a subject array may contain multiple different sets of surface-tethered oligonucleotides, each for detecting the same SNP. In this embodiment, an array may comprise multiple different sets (e.g., 2, 3, 4, 5, 6, 7, 8, 9 or 10 or more sets) of surface-tethered oligonucleotides, where the surface-tethered oligonucleotides of each set are of identical nucleotide sequence except for the SNP nucleotide, where each of the surface-tethered oligonucleotides specifically hybridizes to the same barcode oligonucleotide sequence except for the SNP nucleotide.

In general, methods for the preparation of polynucleotide arrays are well known in the art (see, e.g., Harrington et al, Curr. Opin. Microbiol. (2000) 3:285-91, and Lipshutz et al., Nat. Genet. (1999) 21:20-4) and need not be described in any great detail. The subject oligonucleotide arrays can be fabricated using any means, including drop deposition from pulse jets or from fluid-filled tips, etc, or using photolithographic means. Either polynucleotide precursor units (such as nucleotide monomers), in the case of in situ fabrication, or previously synthesized polynucleotides can be deposited. In some embodiments, the arrays may be constructed to include oligonucleotide analogs such as nucleotide analogs such as 2,6-aminopurines. Such methods are described in detail in, for example U.S. Pat. Nos. 6,242,266, 6,232,072, 6,180,351, 6,171,797, 6,323,043, and U.S. Patent Application US20040086880 A1, etc., the disclosures of which are herein incorporated by reference.

### Utility

The subject method finds use in a variety of applications, where such applications are generally nucleic acid detection applications in which the presence of a particular nucleotide or oligonucleotide in a given sample is detected at least qualitatively, if not quantitatively. In general, any assays involving the use of an oligonucleotide designed to identify the presence of a target analyte may be detected by the subject method. Protocols for carrying out such assays are well known to those of skill in the art and need not be described in great detail here.

Generally, the sample suspected of containing a barcode oligonucleotide, is contacted with surface-tethered oligonucleotides under conditions sufficient for the barcode oligonucleotide to bind to its respective surface-tethered oligonucleotide present on the array. Thus, if the barcode oligonucleotide is present in the sample, it binds to the array and an overhang duplex is formed on the array surface. The overhang duplex is then extended to produce an extended duplex, where the Tₘ is increased compare to the Tₘ of the overhang duplex before extension.

As noted above, a duplex may comprise insufficient complementary regions between the barcode oligonucleotide and the surface-tethered oligonucleotide. In such a case, an overhang duplex would not be extended. For example, if a barcode oligonucleotide nonspecifically hybridizes with features on the array, it would not be extended. If there is no base-pairing between the overhang-adjacent nucleotide and the corresponding nucleotide, there would be extension. Depending on the features of the array, specific surface-tethered oligonucleotides may also be selectively chosen to be subjected to extension by providing specific sets of nucleotides or oligonucleotides to be added onto the ends of barcode oligonucleotides. For example, if the overhang of one set of surface-tethered oligonucleotides comprises a homopolymeric tract of thymines while the overhang of the other set comprises of adenines, only the duplexes comprising the thymine tracts would be extended if there are only adenines or polyadenines in the extension reaction.

Subjecting the overhang duplexes to extension increases the Tₘ of only the duplexes comprising surface-tethered oligonucleotides and barcode oligonucleotides that are correctly matched. The wash conditions may be at a stringency that is suitable for retaining the extended duplexes but separating the non-extended duplexes from the array, as discussed previously. In certain embodiments, where the sample comprising barcode oligonucleotides also comprises long polynucleotides from which the barcode oligonucleotides are cleaved, fragments of genomic DNA or amplifications thereof, other nucleic acids, etc., the subject method may be able to separate these other nucleic acids present in the sample that may add noise to the signal detection from the extended barcode oligonucleotides. This separation may greatly aid in signal detection of barcodes that may otherwise be undetectable without employing the subject method.

Specific analyte detection applications of interest include but not limited to SNP detection assays. One embodiment of SNP detection assays employs structure-specific nucleases such as enzymes with flap endonuclease (FEN) activity to perform an overlap-dependent cleavage assay. In this embodiment, the cleavage of a long polynucleotide depends on specific overlapping structures formed with a target containing SNP, as discussed previously. Cleavage of the polynucleotide to produce a barcode oligonucleotide indicates the presence of the SNP. More details on the specifics of this SNP detection assay can be found in Lyamichev et al. (Lyamichev et al. “Polylmorphism identification and quantitative detection of genomic DNA by invasive cleavage of oligonucleotide probes.” Nat. Biotechnology. (1999) 17:292-296), Stevens et. al (2001), and Mast et al. (2006). In certain embodiments, the sample may include uncleaved polynucleotides and other oligonucleotides used in the overlap-depending cleavage assay in addition to the barcode oligonucleotides of interest. In these embodiments, if the uncleaved polynucleotide comprising the barcode oligonucleotide plus additional sequence is not perfectly complementary to the surface-tethered oligonucleotide 12, the uncleaved polynucleotide may not be extended. In this manner cleaved oligonucleotide barcodes may be discriminated from the uncleaved polynucleotide precursors comprising sequence additional to the oligonucleotide barcode sequence. The subject methods described herein may be able to selectively detect barcode oligonucleotides in such a sample.

Another application of interest may be the detection of ligation products. Several ligation reactions are used to amplify target nucleic acids by ligating primers. In certain embodiments, successful ligation occurs only when base-pairing at nick junction is complementary. In certain cases, presence of ligation products indicates the presence of a specific nucleotide sequence or a SNP. The subject method described herein may be able to selectively detect such ligation products, referenced herein as barcode oligonucleotides, in ligation detection assays or ligation amplification assays. For example, ligation of a particular sequence to a precursor nucleic acid may produce a barcode oligonucleotide which may be extended, while ligation of other sequences to the precursor nucleic acid may produce oligonucleotides which may not be extended. In another embodiment, molecular inversion probe (MIP) detects complementary regions in a target nucleic acid, as a padlock probe. The MIP is linearized and amplified upon detection of such regions. The MIP probes and their tag sequences may also be detected using the subject method. In another embodiment, detection of protein molecules by an antigen conjugated to a barcode oligonucleotide may also be detected using the subject method. More details on these assays can be found in Cao et al. (Cao et al. “Recent developments in ligase-mediated amplification and detection.” Trends in Biotechnology (2004) 22:38-44).

Other assays of interest which may be practiced using the subject method include: genotyping, scanning of known and unknown mutation, gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

The above described applications are merely representations of the numerous different applications for which the subject array and method of use are suited. In certain embodiments, the subject method includes a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In certain embodiments of the subject methods in an array, the array may typically be read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Santa Clara, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of sample analysis, comprising:
a) contacting a surface-tethered oligonucleotide with a sample comprising a barcode oligonucleotide to produce an oligonucleotide duplex comprising a double-stranded surface-proximal region and a single-stranded surface-distal overhang;
b) extending said barcode oligonucleotide using said overhang as a template to produce an extended duplex;
c) subjecting said extended duplex to a wash that separates said oligonucleotide duplex but does not separate said extended duplex; and
d) detecting said extended duplex.

2. The method of claim 1, wherein said wash comprises conditions that preferentially separate said oligonucleotide duplex as compared to said extended duplex.

3. The method of claim 1, wherein said contacting comprise a temperature lower than the Tₘ of said oligonucleotide duplex.

4. The method of claim 1, wherein said extended duplex has a Tₘ that is 4 to 20° C. higher than the Tₘ of said oligonucleotide duplex.

5. The method of claim 1, wherein said overhang comprises 2 to 10 nucleotides.

6. The method of claim 1, wherein said extending comprises contacting said oligonucleotide with a reagent mix comprising a template-dependent polymerase and at least one labeled nucleotide.

7. The method of claim 6, wherein said polymerase is a thermostable polymerase.

8. The method of claim 1, wherein said overhang comprises nucleotide analogs.

9. The method of claim 1, wherein said extending comprises contacting said oligonucleotide duplex with a reagent mix comprising a ligase and a labeled oligonucleotide that is complementary to said overhang.

10. A method of sample analysis comprising:
a) contacting an array of surface-tethered oligonucleotides with a sample comprising barcode oligonucleotides to produce a template array comprising overhang duplexes, each of which comprises a double-stranded variable region proximal to the surface and a single-stranded constant region distal to the surface;
b) subjecting said template array to primer extension conditions to produce an array comprising:
i. extended duplexes comprising extended barcode oligonucleotides; and
ii. non-extended duplexes comprising non-extended barcode oligonucleotides;
c) subjecting said array to a wash that removes said non-extended barcode oligonucleotides but not said extended barcode oligonucleotides from said array; and
d) reading said array to detect said extended duplexes.

11. The method of claim 10, wherein a barcode oligonucleotide of said overhang duplexes comprises an overhang-adjacent nucleotide that is extended only if said overhang-adjacent nucleotide is complementary to the corresponding nucleotide in said surface-tethered oligonucleotide.

12. The method of claim 11, wherein the overhang-adjacent nucleotide is a SNP nucleotide.

13. The method of claim 10, wherein said wash comprises conditions that preferentially separate said non-extended duplexes relative to said extended duplexes.

14. The method of claim 10, wherein said contacting comprises a temperature lower than the Tₘ of said overhang duplexes.

15. The method of claim 10, wherein said extended duplexes have a Tₘ of 4 to 20° C. higher than the Tₘ of said non-extended duplexes.

16. The method of claim 10, wherein said overhang comprises 2 to 10 nucleotides.

17. The method of claim 10, wherein said extended barcode oligonucleotides are Tₘ-matched.

18. A method comprising:
a) performing an overlap-dependent cleavage assay on a target nucleic acid to produce a barcode oligonucleotide product; and
b) analyzing said barcode oligonucleotide product using claim 1.

19. A method of claim 18, wherein said overlap-dependent cleavage assay comprises:
a) contacting a set of oligonucleotides with a target nucleic acid to produce a complex; and
b) cleaving said complex using a flap endonuclease.

20. An array comprising a plurality of surface-tethered oligonucleotides, each of which comprises:
a surface-proximal variable region complementary to a barcode oligonucleotide and
a surface-distal constant region of 4 to 10 nucleotides
wherein said each of surface-tethered oligonucleotides has a length of at least 15 nucleotides.
